(19) United States
(12) Patent Application Publication
(10) Pub. No.: US 2016/0263395 A1
(43) Pub. Date: Sep. 15, 2016

(54) Modular Low-Level Light Therapy System Employing Semiconductor Light Sources

(71) Applicant: FLEXLITE CORPORATION, Tucson, AZ (US)

(72) Inventors: Lawrence Siegel, Menlo Park, CA (US); James Harrison, Oro Valley, AZ (US); Paul Nahass, Cambridge, MA (US); Oscar Romero, Tucson, AZ (US); Gregory Quarles, Oro Valley, AZ (US); Brian Swienton, Dover, NH (US)

(21) Appl. No.: 15/032,308

(22) PCT Filed: Jun. 23, 2015
(86) PCT No.: PCT/US2015/037272

§ 371 Date: Apr. 26, 2016

Related U.S. Application Data
(60) Provisional application No. 62/016,432, filed on Jun. 24, 2014.

(51) Int. Cl. A61N 5/06; A61B 5/021; A61B 5/00; A61B 5/0402; A61B 5/145; A61B 5/01; A61B 5/024; A61B 5/08

(52) U.S. Cl. CPC A61N 5/0616; A61B 5/024; A61B 5/021; A61B 5/08; A61B 5/0402; A61B 5/14542; A61B 5/01; A61B 5/4836; A61B 2562/0219; A61N 2005/0626; A61N 2005/0652; A61N 2005/067

(57) ABSTRACT

The present application discloses various embodiments of a modular low-level light therapy system configured to be worn by or otherwise affixed to the patient. Moreover, unlike prior art systems, the present modular low-level light therapy system disclosed herein may be worn by a moving patient while receiving treatment. Further, the modular low-level light therapy system disclosed herein may easily be adapted from use in various locations on the body of the patient.

### CROSS REFERENCE TO RELATED APPLICATIONS

The present application claims priority to U.S. Provisional Patent Application Ser. No. 62/016,432, filed on Jun. 24, 2014, the entire contents of which are hereby incorporated by reference herein.

### BACKGROUND

Presently, laser light is employed in a number of therapeutic applications for the treatment of mammals. For example, low-level light therapy is commonly used for pain management, to reduce inflammation, and to stimulate photo-biological response to enhance physiological reactions. Typically, appliances and systems used in low-level light therapy applications employ semiconductor Light Emitting Diodes (LEDs) and/or edge-emitting semiconductor lasers to generate optical outputs at wavelengths in the visible and/or near infrared spectral regions.

Generally, low-level light therapy processes (also known as photobiomodualtion therapy) require the non-invasive application of light to the skin of the patient proximate to a treatment area at a sufficient energy and wavelength configured to generate the desired therapeutic response. Ideally, the wavelength and power of the light incident on skin of the patient is sufficient to initiate photo-stimulation while not resulting in dermal or sub-dermal ablation or undesirable thermal degradation and/or heating of the tissue. Presently, low-level light therapy systems utilize a large treatment device which is either strapped to the patient or held by a healthcare provider proximate to the area of treatment. Typically, the patient is required to remain stationary during the treatment process, which may range from several minutes to hours.

While presently available low-level light therapy systems have proven somewhat useful in the past, a number of shortcomings have been identified. For example, presently available systems require the patient to remain substantially stationary and immobile during treatment procedures. As such, this inconvenience may result in the patient foregoing needed treatment. Moreover, requiring a human patient to remain stationary during treatment may pose a substantial inconvenience; however, requiring other mammals to remain stationary during treatment may prove difficult if not impossible without sedation or other means. Further, presently available systems tend to be large, complex, and expensive, and thus, are more adapted for use in professional healthcare facilities.

In light of the foregoing, there is on ongoing need for a less expensive low-level light therapy system adapted to be worn by the patient without requiring the patient to be immobile.

### SUMMARY

The present application discloses various embodiments of a modular low-level light therapy system configured to be worn by or otherwise affixed to the patient. Moreover, unlike prior art systems, the present modular low-level light therapy system disclosed herein may be worn by a moving patient while receiving treatment. Further, the modular low-level light therapy system disclosed herein may easily be adapted from use in various locations on the body of the patient.

In one embodiment, the present application discloses a modular low-level light therapy system and includes one or more device bodies configured to be positioned on the body of a user proximate to a treatment area. In one embodiment, the device bodies are manufactured from compliant materials configured to be easily adapted to conform to the body of a patient. Optionally, the device bodies need not be manufactured from compliant materials or otherwise adapted to be positioned on or in proximity to a treatment area. Further, the modular low-level light therapy system includes one or more light sources positioned on each device body, the light sources configured to output one or more therapeutic optical signals to the treatment area. In addition, the modular low-level light therapy system includes one or more treatment controllers positioned on each device body, the treatment controllers having at least one device circuit therein. The device circuit is in communication with the light sources positioned on the device body and configured to provide at least one control signal to the light sources to regulate the operation of at least one emitter of the light sources. Further, one or more sensors in communication with at least one device circuit and configured to measure one or more treatment parameter from an area of interest of a body of a patient may be included on or in communication with the modular low-level light system. Lastly, the modular low-level light therapy system includes at least one system controller in communication with at least one device circuit and the sensors. The system controller may be configured to vary the operation of the light source via the device circuits of the treatment controllers based on data received from the sensors.

In another embodiment, the present application discloses a modular low-level light therapy system having a distributed control architecture and includes two or more device bodies configured to be positioned on the body of a user proximate to a treatment area. One or more light sources positioned are included on each device body and configured to output one or more therapeutic optical signals to the treatment area. The light sources may comprise an array of multiple semiconductor emitters. Further, the modular low-level light therapy system includes one or more sensors configured to measure one or more treatment parameters from an area of interest of a body of a patient. Lastly, the present embodiment includes one or more treatment controllers positioned on each device body. The treatment controllers have at least one device circuit therein, the device circuits in communication with light sources and device circuits positioned proximate thereto. During operation, the device circuits are configured to provide at least one control signal to the light sources to regulate the operation of at least one emitter of the light sources to vary the operation of the light sources based on data received from the sensors.

Other features and advantages of the modular low-level light therapy systems employing semiconductor light sources as described herein will become more apparent from a consideration of the following detailed description.

### BRIEF DESCRIPTION OF THE DRAWINGS

Various embodiments of a modular low-level light therapy systems employing semiconductor light sources will be explained in more detail by way of the accompanying drawings, wherein:

FIG. 1 shows an elevated perspective view of an embodiment of a wearable low-level light therapy system having a light source and a circuit positioned on a device body;

FIG. 2 shows a schematic of an embodiment of an illumination system having two circuits controlling emitters of a light source used in a wearable low-level light therapy system;

FIG. 3 shows an elevated perspective view of an embodiment of a wearable low-level light therapy system wherein the illumination system is selectively attached to a device body;

FIG. 4 shows a planar perspective view of an embodiment of a skeletal brace incorporating one or more wearable low-level light therapy systems therein;

FIG. 5 shows an elevated perspective view of another embodiment of a brace incorporating one or more wearable low-level light therapy systems therein;

FIG. 6 shows an elevated perspective view of another embodiment of a brace incorporating one or more wearable low-level light therapy systems therein;

FIG. 7 shows an elevated perspective view of an embodiment of a garment incorporating one or more wearable low-level light therapy systems therein;

FIG. 8 shows an elevated perspective view of another embodiment of a garment incorporating one or more wearable low-level light therapy systems therein;

FIG. 9 shows a cross-sectional view of an embodiment of a wearable low-level light therapy system during use;

FIG. 10 shows an elevated perspective view of an embodiment device body or pod of a modular low-level light therapy system;

FIG. 11 shows side planar view of an embodiment device body or pod of a modular low-level light therapy system;

FIG. 12 shows top planar view of an embodiment device circuit of a modular low-level light therapy system;

FIG. 13 shows schematic diagram of a modular low-level light therapy system having an external controller;

FIG. 14 shows schematic diagram of a modular low-level light therapy system having a distributed control architecture in communication with an external controller;

FIG. 15 shows a perspective view of a device body or pod being inserted into a compressive or positioning garment in preparation of use;

FIG. 16 shows a perspective view of a device body or pod inserted into a compressive or positioning garment during use;

FIG. 17 shows a perspective view of an embodiment of a garment adapted for use with an embodiment of a modular low-level light therapy system disclosed herein;

FIG. 18 shows a perspective view of an embodiment of a modular low-level light therapy system disclosed herein;

FIG. 19 shows a perspective view of another embodiment of a modular low-level light therapy system disclosed herein;

FIG. 20 shows a perspective view of an embodiment of an insole or footbed of a shoe adapted for use with an embodiment of a modular low-level light therapy system disclosed herein;

FIG. 21 shows a perspective view of an embodiment of headgear adapted for use with an embodiment of a modular low-level light therapy system disclosed herein; and

FIG. 22 shows a perspective view of an embodiment of a blanket adapted for use with an embodiment of a modular low-level light therapy system disclosed herein.

### DETAILED DESCRIPTION

The present application discloses various embodiments of a low-level light therapy system configured to be worn by or otherwise affixed to the body of a patient or person receiving therapeutic or enhanced recovery treatment. In one embodiment, the user comprises a patient seeking therapeutic treatment. In another embodiment, the user may be a person seeking improved or enhanced recovery following physical exertion or effort. Moreover, unlike prior art technologies and/or systems, the present low-level light therapy system disclosed herein may be worn by a moving patient while therapeutic treatment is being administered thereto.

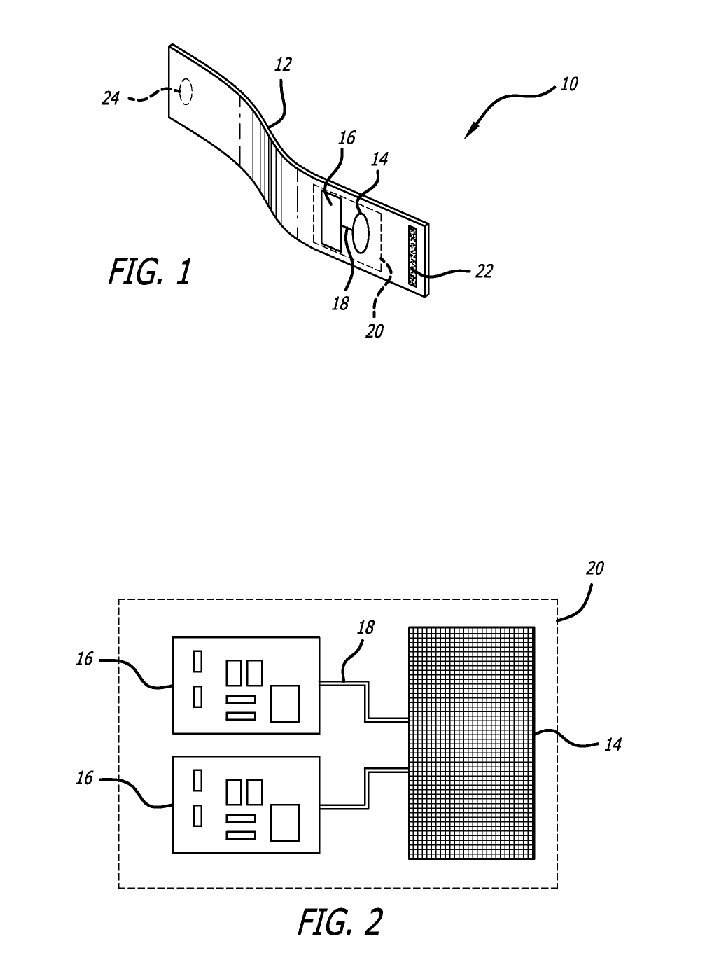

In general, the low-level light therapy systems disclosed herein utilizes at least one semiconductor light source configured to deliver at least one therapeutic optical signal to an area of treatment. As shown in FIGS. 1-3, in one embodiment the low-level light therapy system 10 includes at least one device body 12. The device body 12 may be formed in any variety of shapes and sizes. Further, in one embodiment, the device body 12 may be manufactured from polymer material, although those skilled in the art will appreciate that the device body 12 or any other components of the low-level light therapy system 10 may be manufactured from any variety of biologically compatible materials. Exemplary polymer materials include, without limitations, polyimide, neoprene, polyurethane, polyimide, nylon, and the like. Optionally, the device body 12 may be manufactured from a variety of materials, including, without limitations, polymers, natural fibers (e.g. wool, cotton, bamboo, etc.), silicon, elastomers, and the like. Optionally, the device body 12 may include one or more light delivery devices integrated therein or attached thereto. For example, the device body 12 may include one or more fiber optic devices integrated therein.

Referring again to FIGS. 1-3, at least one semiconductor light source 14 is coupled to device body 12. In one embodiment the light source 14 comprises at least one light emitting diode (hereinafter LED). In an alternate embodiment, the light source 14 comprises at least one laser diode. Exemplary laser diodes configured for use with the present system include, without limitations, edge-emitting laser devices, vertical cavity surface emitting laser devices (hereinafter VCSELs) and the like. Optionally, as shown in FIG. 2, the light source 14 may comprise an array of one or more LEDs or LED die, super-luminescent diode (SLDs), laser diodes or die, or both. Further, in the illustrated embodiment, the light source 14 comprises a LED, a laser diode, or both. For example, LEDs and VCSELs can be fabricated as compact, monolithic arrays of individual emitters to increase the total available power in operation as an ensemble surface-emitting light source. In such cases the individual emitters within an array can be electrically connected to facilitate electrical control of the ensemble as well as integration into flexible/stretchable electronic circuits. Multiple arrays could be similarly connected for ensemble operation and control. Optionally, the light source 14 need not include surface emitting devices. For example, the light source 14 may include one or more fiber optic lasers or fiber optic devices configured to deliver one or more therapeutic signals to various treatment areas. Further, the light source 14 may include any variety of light sources.

In some applications, semiconductor light sources are particularly well suited because of a combination of attributes including: high power-to-volume and high power-to-mass; low voltage and low power requirements; efficient conversion of electrical power to light; compatibility with flexible/stretchable electronic circuits and circuit assemblies; ability to operate at wavelengths of interest for low-level light therapy; reliability (e.g., in terms of expected hours of operation, durability); maturity of the technology and associated means of manufacturing; low cost per unit of light power (e.g., dollars per delivered Watt). In addition, semiconductor light sources offer high spatial coherence, facilitating illumination of remote target areas with minimal or no refractive optics. This is especially true of VCSEL versus edge emitting laser. In addition, these sources have high spectral coherence, concentrating light energy at wavelengths of particular interest for specific low-level light therapy applications. Among semiconductor light sources, those based on III-V compounds including both Gallium and Arsenic are the most commonly used for low-level light therapy applications because of their high efficiency (conversion of electrical power to optical power), spectral compatibility with low-level light therapy applications and low cost.

Referring again to FIG. 1-3, in one embodiment the light source 14 is configured to emit at last one therapeutic optical signal having a wavelength from about 400 nm to about 1500 nm. For example, in one embodiment, the light source 14 is configured to output at least one therapeutic optical signal having a wavelength from about 600 nm to about 1100 nm. In another embodiment, the light source 14 is configured to output at least one therapeutic optical signal having a wavelength from about 700 nm to about 1050 nm. In another embodiment, the light source 14 is configured to output at least one therapeutic optical signal having a wavelength from about 780 nm to about 1000 nm. In yet another application, the light source 14 is configured to output at least one therapeutic signal having a wavelength of about 700 nm to about 800 nm. Optionally, the light source 14 may be configured to output multiple optical signals at a single wavelength or a narrow wavelength range. In another embodiment, the light source 14 may be configured to output any number of optical signals at different wavelengths. For example, the light source 14 may be configured to output a first therapeutic optical signal at a first wavelength and at least a second therapeutic optical signal at at least a second wavelength. Further, the light source 14 may be configured to output a continuous wave optical signal, a pulsed optical signal, and/or both. Further, the light source 14 may include one or more optical elements positioned thereon or proximate thereto to condition or otherwise modify the therapeutic light emitted therefrom. For example, the light source 14 may include one or more filters, gratings, lenses and the like positioned thereon or proximate thereto. For example, the light source 14 may include one or more optical metamaterials in optical communication therewith. Exemplary metamaterials include, without limitations, one or more ENZ (epsilon-near-zero) metamaterials thereby permitting the output of the light source 14 to be widely tunable over a desired range (e.g. all visible wavelengths).

As shown in FIGS. 1-3, the low-level light therapy system 10 includes at least one circuit 16 in electrical communication with the light source 14. In one embodiment, the circuit 16 is configured to provide power to the light source 14. In another embodiment, the circuit 16 is configured to provide data to and receive data from the light source 14. Optionally, the circuit 16 may include one or more semiconductor devices, chips, sensors, controllers, processors, power supplies, batteries, energy sources, voltage regulators, current regulators, user interfaces, display devices, communication devices, user interfaces, wireless devices, MEMS devices, lab-on-a-chip systems, and the like. For example, in some embodiments, the circuit 16 includes one or more sensors configured to provide biological information and/or data received from the treatment area. Optionally, the biological information received from the treatment area may be used to vary the treatment parameters such as the duration of and/or frequency of the treatment, wavelength, pulse length, intensity of the illumination, pulse repetition rate, and the like. In addition, the circuit 16 may include one or more controllers configured to provide information, data, and/or one or more control signals to and receive information, data, and/or one or more control signals from one or more bio-medical sensors, controllers, and the like positioned external the body of the user and/or within the body of a user. For example, the circuit 16 may be in communication with at least one external controller (e.g. a smartphone, handheld device, computer, computer network, processors, and the like) and at least one sensor or similar device positioned on or within the user. Optionally, a circuit 16 positioned on a first low-level light therapy system 10 may be configured to provide data to and receive data from at least a second low-level light therapy system 10 positioned on the patient. As such, the circuit 16 may act as a conduit configured to provide information to and receive information from the external control device and the sensor wirelessly and/or via a conduit. For example, the circuit 16 may be configured to provide and receive data from at least one of the light source 14, control pumps, drug delivery systems, pacemakers, and the like positioned on or within the body of a patient or user.

In addition, any number of additional sensors may be in communication with or included on the circuit 16. Exemplary additional sensors include, without limitation, flow sensors, oxygenation sensors, tissue temperature sensors, accelerometers, force meters, and the like. In one embodiment, the low-level light therapy system 10 includes one light source 14 and one circuit 16. Optionally, the low-level light therapy system 10 may include a single light source 14 in communication with multiple circuits 16. In another embodiment, the low-level light therapy system 10 includes multiple light sources 14 in communication with a single circuit 16. Further, the low-level light therapy system 10 may include multiple light sources 14 in communication with multiple circuits 16.

Further, the circuit 16 may include one or more integrated circuit devices, flexible circuits, and/or assemblies of integrated circuits and/or flexible circuits. Optionally, the circuit 16 may include one or more processors configured to be in communication with at least one external controller (not shown). Exemplary external controllers include, for example, computers, handheld devices such as smart phones, tablet computers, and the like. As such, at least one external processor may be configured to provide data to and/or receive data from at least one of the light source 14, circuit 16, and/or both via the circuit 16.

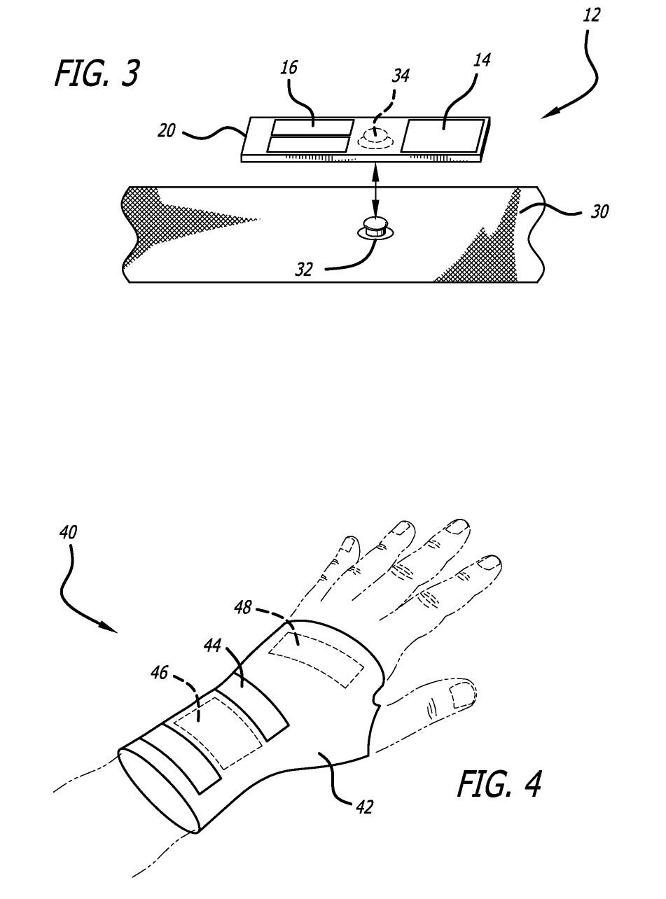

Optionally, as shown in FIGS. 1-3, the light source 14 and the circuit 16 may cooperatively form at least one illumination system body or area 20. In one embodiment, the light source 14 and circuit 16 are integral to the device body 12 of the low-level light therapy system 10. As such, the illumination system body 20 comprises an area containing the light source 14, circuit 16, and the at least one conduit 18 electrically coupling the light source 14 to the circuit 16. In another embodiment, at least one of the light source 14, circuit 16, or both may be detachably coupled to the device body 12. For example, in the embodiment shown in FIG. 3, the illumination system 20 including the light source 14 and circuit 14 are detachably coupled to device body 12. More specifically, the device body 12 may include at least one coupling area 30 formed thereon. In the illustrated embodiment, the coupling area 30 includes at least one coupling feature 32 configured to cooperatively attach to at least one coupling device 34 formed on or otherwise positioned on at least one of the light source 14, circuit 16, and/or illumination system body 20. As such, the illumination system 20 may be removed from the device body 12 in whole or in part, thereby permitting the device body 12 to be washed or otherwise treated (e.g. sterilization, disinfecting, cleaning, and the like) using conventional techniques without damaging the light source 14, circuit 16, conduits 18, and/or illumination system 20. Further, at least one of the light source 14, circuit 16, conduits 18, and/or illumination system 20 may include various housings or other devices to prevent environmental damage to the various components of the low-level light therapy system 10.

Referring again to FIGS. 1-3, in one embodiment the various components of the illumination system 20 may incorporate flex or stretchable electronic circuit technology. More specifically, flexible electronic circuits are by definition compatible with some degree of mechanical deformation. Commonly, flexible circuits are formed by mounting electronic components (e.g. the light source 14 and/or the circuit 16) on flexible substrates, with entire assemblies consisting of one or more (e.g., multi-layer) substrates. As such, at least one of the light source 14, circuit 16, conduit 18, and/or illumination system 20 may be mounted on at least one flexible substrate or may form a flexible electronic circuit. In the present application flexible electronic circuits are particularly useful when intended for deployment within, or as part of, wearable garments and/or accessories (e.g., bracelets). The flexibility of these circuits and the illumination system 20 can be enhanced both by the selection of substrate materials and the design and selection of embedded components, electrical interconnects and mechanical structures forming the illumination system 20. As such, in one embodiment, the flexible circuits may be integrated into various garments, sleeves, braces, wraps, hats, and the like. Further, the effectivity of the low-level light therapy system 10 may also be enhanced by optimizing the design of the low-level light therapy system 10 for use with of one or more garments, accessories, and/or attachment systems or mechanisms (e.g. tape, kinesiology tape, wraps, sleeves, braces, and the like). Optionally, the low-level light therapy system 10 may be configured for use with re-usable garments or disposable garments. For example, in one embodiment the low-level light therapy system 10 is configured for use with compressive garments, thereby providing therapeutic light therapy while simultaneously providing therapeutic compressive support. As such, in addition to providing compressive support, the compressive force of the compressive garment may securely position the low-level light therapy system 10 proximate to a treatment area on a user. In another embodiment, the low-level light therapy system 10 may be configured for use with disposable bandages, wraps, diapers, patches, and the like.

Optionally, one or more portable energy sources may be included within or otherwise coupled to the illumination system 20. For example, in one embodiment at least one power supply system is included within circuit 16 of the illumination system 20. Exemplary power supply systems include, for example, batteries. In one embodiment, the power supply system may be rechargeable. As such, the power supply system may be recharged by conventional means through a wired connection (e.g., utilizing a standardized connector such as a micro USB port) or via some form of wireless charging wherein the receiving antenna and conversion electronics are part of or in communication with the circuit 16. In fact, energy sourced from an external source separate from the low-level light therapy system could be transported wirelessly to directly supply some or all of the devices, components and sub-assemblies of the low-level light therapy system in combination with or in lieu of batteries.

As shown in FIG. 1, the at least one attachment device 22 is coupled to, positioned on, or otherwise formed in the device body 12 of the low-level light therapy system 10. For example, in one embodiment, the attachment device 22 comprises hook and loop material thereby permitting the user to couple the low-level light therapy system 10 to the body of the user such that the light emitted from the light source 14 will be directed into the body of the user proximate to an area of interest or treatment area. Those skilled in the art will appreciate that any number and variety of attachment devices 22 may be used with the low-level light therapy system 10.

Referring again to FIG. 1, the low-level light therapy system 10 may further include one or more additional therapeutic systems 24 coupled to the device body 12, light source 14, circuit 16, and/or illumination system 20. Exemplary additional therapeutic systems include, without limitations, muscle stimulations systems, compression systems, chillers/cooling elements, heaters, pumps, drug-delivery systems, pacemakers, ultrasound systems, diagnostic systems, monitoring systems and the like.

FIGS. 4-8 show the various embodiments of the low-level light therapy system 10 disclosed herein incorporated into various braces and garments. For example, FIG. 4 shows an embodiment of a skeletal brace 40 configured to be applied to the wrist of a user to deliver therapeutic light to a treatment area. As shown, the brace 40 includes a brace body 42 having at least one attachment device 44 thereon. Further, the brace 40 includes at least one low-level light therapy system coupled thereto or included thereon. In the illustrated embodiment, a first low-level light therapy system 46 and a second low-level light therapy system 48 are positioned on the brace body 42 and configured to direct therapeutic light into the wrist of the user when worn by the user. Unlike prior art systems, the user of the brace 40 shown in FIG. 4, which includes the low-level light therapy system 46, 48, is not required to remain stationary. Rather, the user may perform substantially normal functions required in activities of daily life.

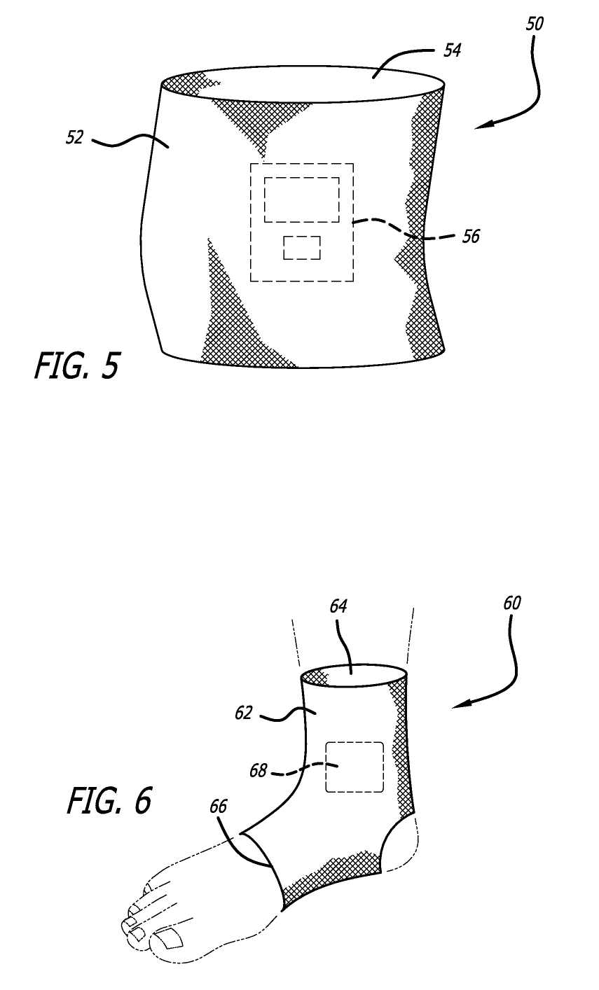

Similarly, FIG. 5 shows an embodiment of brace 50 configured to receive at least one body part therein. For example, the brace 50 shown in FIG. 5 may be configured for use on fingers, wrists, forearms, elbows, biceps, shoulders, triceps, hamstrings, quadriceps, knees, calves, toes, and the like. As shown, the brace 50 includes a brace body 52 defining at least one passage 54. Further, one or more low-level light therapy systems 56 may be coupled to or otherwise positioned on the brace 50 and configured to deliver therapeutic low-level light therapy to an area of interest. In the illustrated embodiment, the brace body 52 may be manufactured from spandex, polyurethane, neoprene, polyimide, or other compressive material and/or material combinations or blends configured to securely position and retain the low-level light therapy system 56 at a desired location.

FIG. 6 shows still another embodiment of the low-level light therapy system incorporated into a skeletal brace. As shown, the ankle brace 60 includes brace body 62 defining a first passage 64 sized to receive the low leg of the user and a second passage 66 sized to receive the foot of the user. Further, at least one low-level light therapy system 68 is coupled to or otherwise included on the brace 60 and configured to deliver therapeutic low-level light therapy to an area of interest. Like the previous embodiment, the brace body 62 may be manufactured from spandex, polyurethane, neoprene, polyimide, or other compressive material configured to securely position and retain the low-level light therapy system 68 at a desired location.

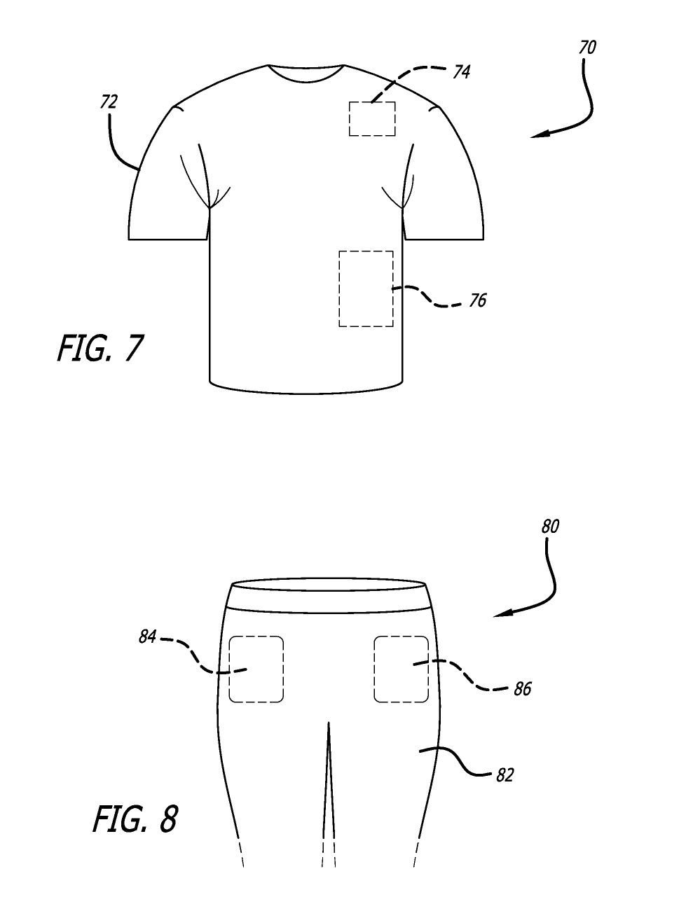

FIG. 7 shows another embodiment of the low-level light therapy system incorporated into a shirt and configured to deliver low-level light therapy to an area of interest located on the upper torso and/or shoulder of the user. As shown, the shirt 70 includes a shirt body 72 having at least one low-level light therapy system coupled thereto or included thereon. In the illustrated embodiment, a first one low-level light therapy system 74 and a second one low-level light therapy system 76 are detachably coupled to the shirt 70. During use, the user would couple, either by wire or wireless connection, the one low-level light therapy systems 74, 76 to the shirt using any variety of attachment devices (See FIG. 1, attachment device 22). Thereafter, the user would initiate the treatment process. For example, in one embodiment, the user would couple the one low-level light therapy system 74, 76 to a user control device (e.g. a handheld device, tablet computer, smartphone, etc.), select the treatment program and parameters from an application, programs or similar control software, and initiate and/or control the treatment process. Thereafter, while the treatment process is occurring, the user may continue his normal activities without being required to remain substantially stationary. In one embodiment, the shirt 70 is manufactured from spandex, polyurethane, neoprene, polyimide, or other compressive material configured to securely position and retain the low-level light therapy system 74, 76 at a desired location.

FIG. 8 shows another embodiment of the low-level light therapy system incorporated into a pants and/or shorts and configured to deliver low-level light therapy to an area of interest located on the lower torso of the user. As shown, the shorts 80 include a body 82 having at least one low-level light therapy system coupled thereto or included thereon. In the illustrated embodiment, a first one low-level light therapy system 84 and a second one low-level light therapy system 86 are detachably coupled to the shorts 80. During use, the user would couple the one low-level light therapy systems 84, 86 to the shorts using any variety of attachment devices (See FIG. 1, attachment device 22). Thereafter, the user would initiate the treatment process. For example, in one embodiment, the user would couple, either by wire or wireless connection, the one low-level light therapy system 84, 86 to a user control device (e.g. a handheld device, tablet computer, smartphone, etc.), select the treatment program and parameters from an application, programs or similar control software, and initiate the treatment process. Like the previous embodiment, while the treatment process is occurring, the user may continue his normal activities without being required to remain substantially stationary. In one embodiment, the shorts are manufactured from spandex, polyurethane, neoprene, polyimide, or other compressive material configured to securely position and retain the low-level light therapy system 84, 86 at a desired location.

As shown in FIGS. 4-8, the low-level light therapy system disclosed herein may be attached to or otherwise incorporated into any number of garment, braces, and the like. Exemplary garments include, without limitations, shirts, pants shorts, socks, headbands, skull caps, scarves, hats, caps, gloves, and the like. Similarly, the low-level light therapy system disclosed herein may be include within or coupled to skeletal splints, braces, sleeves, functional orthopedic braces (e.g. CTI-type devices), cervical collars, back braces, and the like. Further, the low-level light therapy system may be included within or coupled to various bandages, wraps, braces and the like used on mammals. As such, the low-level light therapy system disclosed herein may be easily configured to deliver a therapeutic treatment to various limbs, in whole or in part, joints, musculature, and the skeletal structure of a patient.

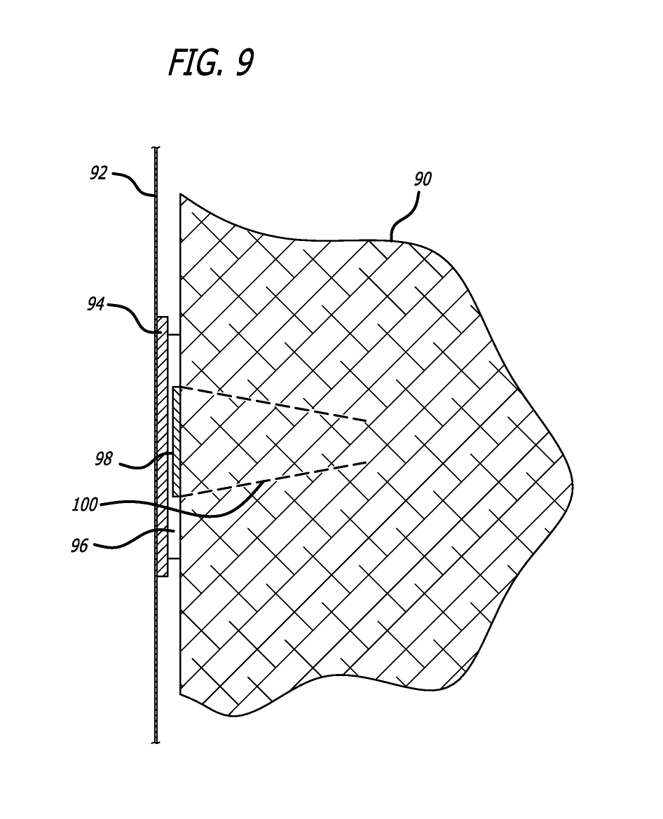

FIG. 9 shows an embodiment of a low-level light therapy system disclosed in the present application during use. As shown, the garment 92 (e.g. shirt) is worn by the user. In one embodiment, the garment comprises a compression shirt configured to provide support compressive pressure to the musculature 90 of the user. At least one low-level light therapy system 94 is detachably coupled to the garment 92. As detailed above, the low-level light therapy system 94 includes at least one flexible circuit 96 in communication with at least one light source 98 configured to emit at least one optical signal 100 at a wavelength configured to stimulate a photo-biological response within the musculature 90 and/or other body constituent of the human and/or animal user. As stated above, the compressive force applied by the garment 92 may be sufficient to maintain the low-level light therapy system 94 at a desired location during the treatment process.

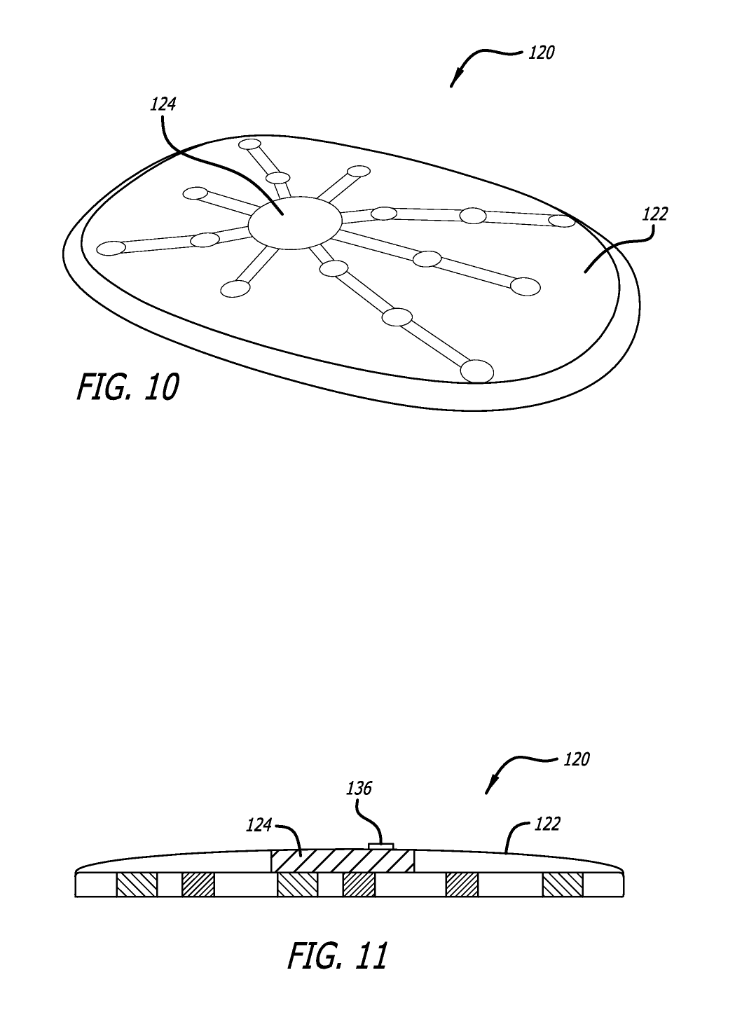

FIGS. 10-12 shows various views of an embodiment of a modular low-level light therapy system configured to deliver at least one therapeutic optical signal to an area of treatment. As shown, the modular low-level light therapy system 120 includes one or more device bodies, pods, or substrates 122 having at least one treatment device circuit 124 positioned thereon or supported by the device body 122. As such, the modular low-level light therapy system may be formed from one or more pods 122 positioned proximate to an area of interest. In one embodiment, at least one device body 122 used in the modular low-level light therapy system comprises a compliant substrate. Optionally, at least one device body 122 used in the modular low-level light therapy system may comprise a rigid substrate. Further, in one embodiment, the device body 122 is manufactured from a biologically compatible material, including, without limitations, polyimide, neoprene, polyurethane, polyimide, nylon, and the like. In another embodiment, the device body 122 is manufactured using three-dimensional printing techniques thereby permitting the patient to receive a custom device body 122 almost in minutes. As such, the device body 122 may be made from any variety of materials configured to be three-dimensionally printed. Optionally, the device body 122 may be manufactured from a variety of materials, including, without limitations, polymers, natural fibers (e.g. wool, cotton, bamboo, etc.), silicon, elastomers, and/or similar biologically compatible materials. Further, those skilled in the art will appreciate that the device body 122 may be manufactured from any variety of materials permitting washing, sterilization, and the like.

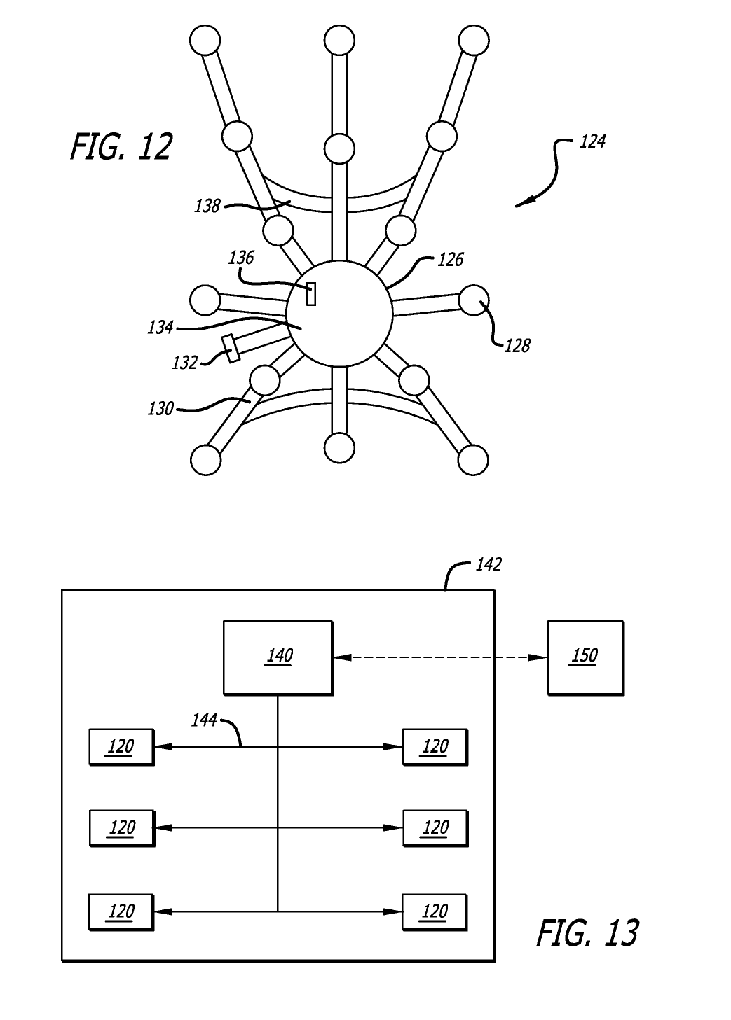

Referring again to FIGS. 10-12, one or more treatment device circuits 124 may be positioned on, integrated within, or otherwise coupled to the device body 122 forming the low-level light therapy system 120. For example, in one embodiment, at least a portion of the treatment device circuit 124 is positioned within the device body 122. Further, at least a portion of the treatment device circuit 124 may traverse through at least a portion of the device body 122. As shown in FIG. 12, in one embodiment, the treatment device circuit 124 includes at least one treatment device controller 126 in communication with at least one low-level light source 128. The treatment device controller 126 may be configured to receive data from and transmit data to at least one of controller coupled thereto or in communication therewith. Further, the treatment device controller 126 may be configured to provide energy to the light sources 128 or other device coupled thereto. In one embodiment, at least one light source is detachably coupled to the treatment device controller. In another embodiment, at least one light source is non-detachably coupled to the treatment device controller. As such, the treatment device controller 126 may include one or more power supplies, batteries, and the like therein. Further, in one embodiment the light source 128 comprises at least one light emitting diode (hereinafter LED). In an alternate embodiment, the light source 128 comprises at least one laser diode. Exemplary laser diode configured for use with the present system include, without limitations, edge-emitting laser devices, vertical cavity surface emitting laser devices (hereinafter VCSELs) and the like. Optionally, the light source 128 may comprise an array of one or more LEDs or LED die, super-luminescent diodes (SLDs), laser diodes or die, or both. Further, in the illustrated embodiment, the light source 128 comprises a LED, a laser diode, an edge-emitting laser device, SLDs, or any combination of the aforementioned light sources. For example, LEDs and VCSELs can be fabricated as compact, monolithic arrays of individual emitters to increase the total available power in operation as an ensemble surface-emitting light source. In such cases the individual emitters within an array can be electrically connected to facilitate electrical control of the ensemble as well as integration into flexible/stretchable electronic circuits. Multiple arrays could be similarly connected for ensemble operation and control. Optionally, the light source 128 need not include surface emitting devices. Optionally, the light source 128 may include one or more fiber optic lasers or fiber optic devices configured to deliver at least one therapeutic signal to various treatment areas. Further, the light source 128 may include any variety of light sources.

As shown in FIG. 12, one or more conduits 130 may couple the treatment device controller 126 to one or more light sources 128. In one embodiment, the conduits 130 comprise flexible device configure to compliantly couple the various components of the treatment device circuits 124 together electrically. Further, the treatment device circuit 124 may include one or more sensors, user-interface devices, or other components 132 configured to receive or provide information to and from the treatment device controller 126. For example, in one embodiment, the sensor 132 may comprise one or more thermocouples configured to measure dermal or sub-dermal tissue temperature. In another embodiment, the sensor 132 may comprise one or more accelerometers configured to measure tissue movement, muscle extensions/contraction, muscle twitch, and the like. In still another embodiment, the sensor 132 may comprise one or more oxygen sensors configured to measure blood or tissue oxygenation. Optionally, various other sensors may be used in the present system, including, without limitations, heart rate sensors, blood pressure sensors, respiration sensors, GPS devices, EEG devices, ECG, devices, biomedical sensors, cryotherapy systems, chemical sensors, small molecule sensors, ions sensitive devices and materials, protein sensing devices and materials, lipid sensing devices and materials, and force meters and other sensors know in the art. Like the light sources 128, the sensors 132 may be coupled to the treatment device controller 126 via at least one conduit 130. For example, in one embodiment the user and/or clinician may be selectively coupled any number of conduits 130 having any variety or number of light sources 128 and sensors 132 to the treatment device controller 126. Further, the conduit 130 may be used to couple multiple treatment device controllers 126 together, each treatment device controller 126 having any number of light sources 128 and sensors 132 coupled thereto, thereby forming a treatment matrix or network. Optionally, numerous treatment device controllers 126 may be coupled together wirelessly.

Referring again to FIG. 12, at least one coupler 136 may be positioned on or proximate to the body 134 of the treatment device controller 126. The coupler 136 may be configured to couple the treatment device controller 126 to a power source thereby permitting at least one battery of rechargeable power supply within the treatment device controller 126 to be recharged. In another embodiment, the device controller 126 may be configured to be wirelessly recharged. Optionally, one or more of the conduits 130 having one or more light sources 128 positioned thereon may be coupled to the treatment device controller 126 via at least one coupler 136. As such, the shape and transverse dimensions of the device circuit may be easily customized to provide optimal therapeutic treatment to an area of interest. In one embodiment, the coupler 136 comprises a micro-USB coupler, although those skilled in the art will appreciate that any variety of coupler 136 may be used with the present system. Further, optionally, one or more support members 138 may be formed on or otherwise coupled to at least one of the treatment device controller 126, light sources 128, conduit 130, sensors 132, treatment device controller body 134, and coupler 136. In an alternate embodiment, the treatment device body 124 may be manufactured without support members 138.

FIG. 13 shows schematically one embodiment of the therapy system which incorporates multiple modular low-level light therapy systems 120 to provide therapeutic treatment or to aid in recovery of mammals. As shown in FIG. 13, in one embodiment the treatment system 142 includes one or more modular low-level light therapy systems 120 coupled to at least one therapy system controller 140 via at least one conduit 144. In the illustrated embodiment, six (6) modular low-level light therapy systems 120 are used in the treatment system 142, although those skilled in the art will appreciate that any number of modular low-level light therapy systems 120 may be used.

Further, in one embodiment, the treatment system controller 140 may be detachably coupled to the at least one of the modular low-level light therapy system 120, the conduit 144, or both. For example, in one embodiment, the treatment system controller 140 may comprise a detachable dongle thereby permitting the treatment system controller 140 to be selectively attached to and detached from the treatment system 142. Optionally, the treatment system controller 140 need not be detachable from the treatment system 142.

Referring again to FIG. 13, in one embodiment, the treatment system controller 140 is configured to receive data from and provide data to at least one of the external controller 150, modular low-level light therapy systems 120, or both. Further, the treatment system controller 140 may include at least one power supply, battery, or the like therein, the power supply configured to provide power to the modular low-level light therapy systems 120 of the treatment system 142 via the conduit 144. For example, in one embodiment, the treatment system controller 140 includes at least one rechargeable battery therein, thereby permitting the user to selectively detach the treatment system controller 140 from the treatment system and recharge the battery located therein.

Optionally, the treatment system controller 140 may include any number of other devices therein, including, without limitations, various sensors, accelerometers, heart rate monitors, blood pressure monitors, oxygenation monitors, temperature sensors, heating devices, user interface devices, displays, GPS devices, tactile alert devices, audio devices, one or more semiconductor devices, processors, power supplies, voltage regulators, current regulators, communication devices, cryotherapy systems, wireless devices, MEMS devices, lab-on-a-chip systems, and the like. For example, in one embodiment, the treatment system controller 140 may include one or more audio or tactile alert devices configured to alert the user when treatment has initiated or been completed, when power resources are low, and the like. In another embodiment, the treatment system controller 140 may include one or more accelerometers therein, the accelerometers configured to measure muscle twitch and the like.

As shown in FIG. 13, at least one conduit 144 may be used to couple the modular low-level light therapy systems 120 to the treatment system controller 140. In one embodiment, the conduits 144 are configured to have at least one modular low-level light therapy system 120, treatment system controller 140, or both detachably coupled thereto. Further, in one embodiment, the conduits 144 comprise flexible/stretchable conduits configured to be integral to at least one fabric or garment. Exemplary garments include, without limitations, shirts, pants shorts, socks, headbands, hats, caps, skull caps, scarves, gloves, and the like. For example, the conduit 144 may be woven into, or otherwise not detachably coupled to the garment. In another embodiment, at least one of the treatment system 142, treatment system controller 140, conduit 144, and/or modular low-level light therapy system 120 may be positioned proximate to an area of treatment by at least one of a compressive wrap, tape, or strap, compressive garment, compressive sleeve, or biologically-compatible adhesive. In another embodiment, the flexible conduit 144 may be non-detachably coupled to garment.

Similarly, the conduit 144 may be integral to or coupled to skeletal splints, braces, sleeves, functional orthopedic braces (e.g. CTI-type devices), cervical collars, back braces, and the like. Further, the conduit 144 may be included within or coupled to various bandages, wraps, blankets and the like used on mammals. Optionally, the conduit 144 may be detachably coupled to various skeletal splints, braces, sleeves, cervical collars, back braces, bandages, wraps, blankets and the like.

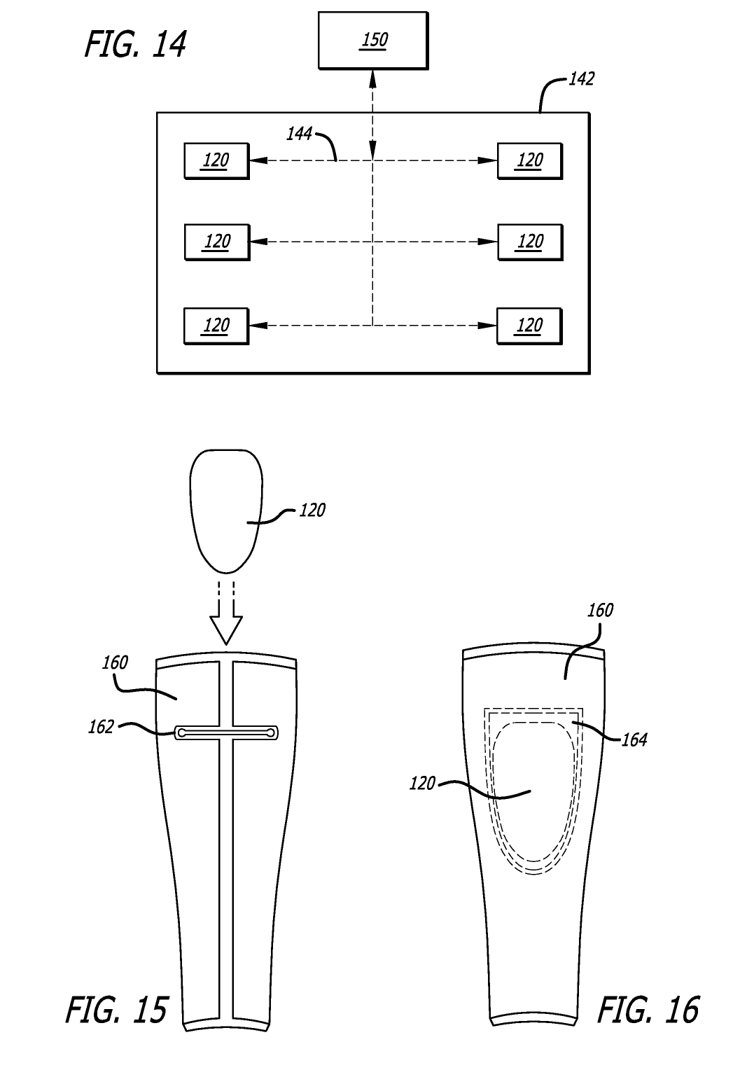

FIG. 14 shows an alternate embodiment of a therapy system which incorporates multiple modular low-level light therapy systems 120 to provide therapeutic treatment or to aid in recovery of mammals. Like the previous embodiment, the therapy system shown in FIG. 14 includes multiple modular low-level light therapy systems 120, although, like the previous embodiment, those skilled in the art will appreciate that the therapy system may be configured to include a single modular low-level light therapy system 120. However, in the present embodiment, the treatment system 142 utilizes a distributed control system, wherein the individual modular low-level light therapy systems 120 cooperatively form a treatment system control architecture thereby forgoing the need for the treatment system controller 140 shown in FIG. 13. As such, the individual modular low-level light therapy systems 120 may be configured to provide data to and receive data from neighboring modular low-level light therapy systems 120, the external controller 150, or both. In one embodiment, the modular low-level light therapy systems 120 may communicate with associated modular low-level light therapy systems 120, the external controller 150, or both wirelessly. In the alternative, the modular low-level light therapy systems 120 may communicate with associated modular low-level light therapy systems 120, the external controller 150, or both via at least one conduit. In an alternate embodiment, the compressive sleeve 120 may be manufactured without a device receiver 162 and device pocket 164. Rather, the compressive force applied by the compressive sleeve 160 may be sufficient to position and retain the modular low-level light therapy system 120 proximate to an area of treatment.

FIGS. 15-22 show various embodiments of the modular low-level light therapy system 120 in use. For example, as shown in FIGS. 15 and 16, a compressive sleeve 160 may be configured to receive at least one therein. FIG. 15 shows a perspective view of the sleeve 160 which includes at least one device receiver 162 sized to receive at least one modular low-level light therapy system 120 therein formed thereon. FIG. 16 shows a cross-sectional view of the sleeve 160 which includes at least one device pocket 164 formed or otherwise attached thereto. In one embodiment, the device pocket 164 is manufactured from a mesh material. In another embodiment, the device pocket 164 is manufactured from any material substantially transparent to an optical signal having a wavelength in the range from about 200 nm to about 2000 nm. The device pocket 164 is in communication with the device receiver 162. During use, the user inserts the modular low-level light therapy system 120 into device pocket 164 via the device receiver 162 and dons the sleeve. Thereafter, the user positions the sleeve such that the modular low-level light therapy system 120 is positioned proximate to an area of treatment. Finally, the user initiates treatment via at least one of the treatment system controller 140, if present, the external controller 150, if present, or both. (See FIGS. 13 and 14).

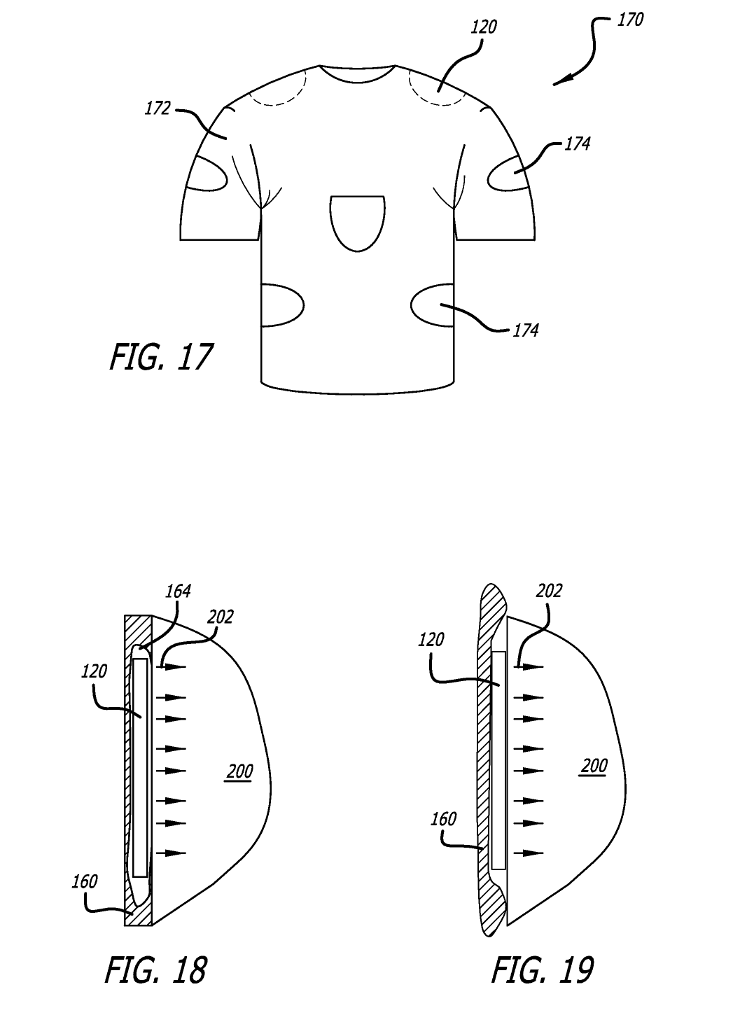

FIG. 17 shows an alternate application of the modular low-level light therapy system 120. As shown in FIG. 17, a shirt 170 having garment body 172 may be configured to receive multiple modular low-level light therapy systems 120 therein. As shown, the garment body 172 may include one or more device pockets 174 sized to receive at least one modular low-level light therapy system 120 therein. In the alternative, like the previous embodiment, the shirt 170 may be configured to provide sufficient compressive or positioning force to the modular low-level light therapy system 120 to negate the need for device pockets 174. FIG. 17 shows at least one modular low-level light therapy system 120 compressively positioned proximate to an area of treatment, the compressive force being applied to the modular low-level light therapy system 120 by the garment body 172.

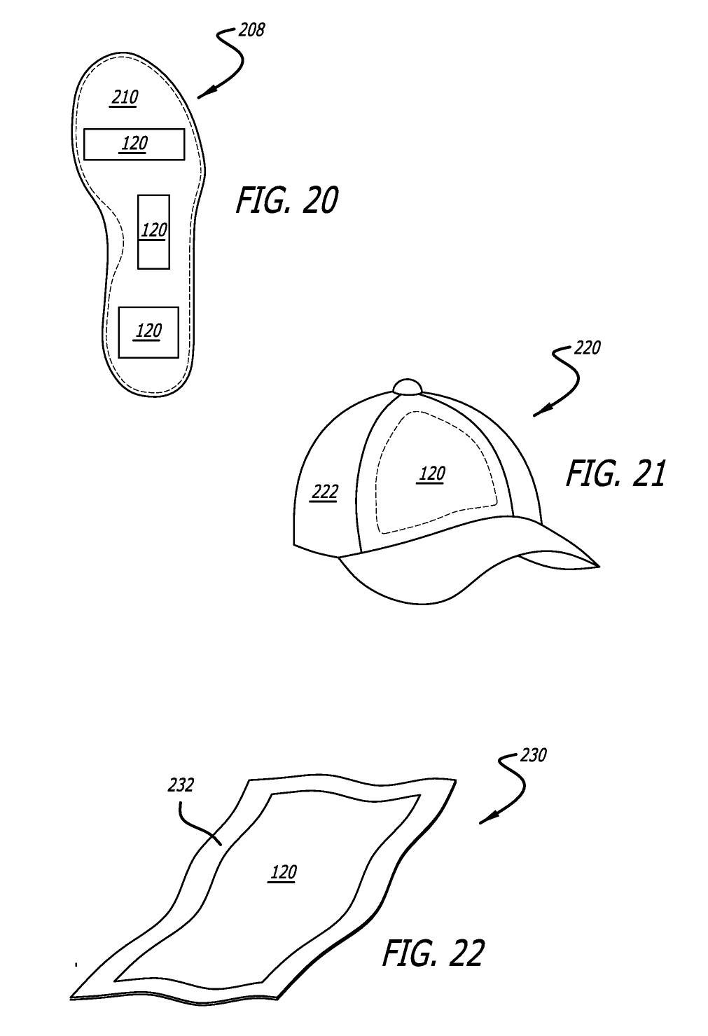

FIGS. 18 and 19 show cross-sectional views of various embodiments of the modular low-level light therapy system 120 in use. As shown in FIG. 18, the modular low-level light therapy system 120 may be positioned within the device pocket 164 formed within the sleeve 160. See FIGS. 15 and 16. Thereafter, the sleeve 160 may be positioned on the body of the user or patient proximate to a treatment area 200. During use, the therapeutic treatment 202 is applied through the material forming the device pocket 164. In the alternative, FIG. 19 shows an embodiment of modular low-level light therapy system 120 positioned on the body of the user or patient proximate to an area of treatment 200 and retained in the desired location by the compressive force applied by the sleeve 160. As such, the therapeutic treatment 202 is applied directly to the tissue surface. Those skilled in the art will appreciate that while FIGS. 18 and 19 illustrate the use of the modular low-level light therapy system 120 with a sleeve, the modular low-level light therapy system 120 may be effectively used with any variety of shirts, pants shorts, socks, headbands, hats, skull caps, scarves, caps, gloves, compressive wraps, tapes, straps, compressive garments, compressive sleeves, skeletal splints, braces, sleeves, functional orthopedic braces (e.g. CTI-type devices), cervical collars, back braces, bandages, blankets and the like used on mammals. For example, FIG. 20 shows an insole 208 having an insole body 210 having one or more modular low-level light therapy system 120 positioned therein or coupled thereto. Similarly, FIG. 21 shows an embodiment of a cap 220 having a cap body 222 having one or more modular low-level light therapy system 120 positioned therein or coupled thereto. Further, FIG. 22 shows an embodiment of a blanket 230 having a blanket body 232 having one or more modular low-level light therapy system 120 positioned therein or coupled thereto. While some garments are particularly well suited for use on humans, other applications, such as braces, wraps, compressive sleeves, blankets, and the like are applicable for human use as well as any variety of mammals and animals, including, without limitations, horses, dogs, cats, cows, birds, reptiles, and the like.

The embodiments disclosed herein are illustrative of the principles of the invention. Other modifications may be employed which are within the scope of the invention. Accordingly, the devices disclosed in the present application are not limited to those precisely as shown and described herein.

1. A modular low level light therapy system, comprising:
one or more device bodies configured to be positioned on the body of a user proximate to a treatment area;
one or more light sources positioned on each device body, the light sources configured to output one or more therapeutic optical signals to the treatment area, the light sources comprising of an array of multiple semiconductor emitters;
one or more treatment controllers positioned on each device body, the treatment controllers having at least one device circuit therein, the device circuit in communication with the light sources positioned on the device body and configured to provide at least one control signal to the light sources to regulate the operation of at least one emitter of the light sources;
one or more sensors in communication with at least one device circuit and configured to measure one or more treatment parameter from an area of interest of a body of a patient; and
at least one system controller in communication with at least one device circuit and the sensors, the system controller configured to vary the operation of the light source via the device circuits of the treatment controllers based on data received from the sensors.

2. The modular low level light therapy system of claim 1 wherein at least one device body comprises a compliant substrate configured to engage and retain at least of the light source and treatment controller therein.

3. The modular low level light therapy system of claim 1 wherein at least one device body is manufactured from at least one biologically compatible material selected from the group consisting of polyimide, neoprene, polyurethane, polyimide, nylon, silicon, polymers, elastomers, and natural fibers.

4. The modular low level light therapy system of claim 1 wherein at least one device body is manufactured using three dimensional printing processes.

5. The modular low level light therapy system of claim 1 wherein the light source comprises one or more devices selected from the group consisting of LEDs, super luminescent LEDs, laser diodes, edge-emitting laser devices, vertical cavity surface emitting laser devices, and fiber optic devices.

6. The modular low level light therapy system of claim 1 further comprising multiple light sources positioned on one or more compliant conduits, each conduit coupled to a body of the treatment controller.

7. The modular low level light therapy system of claim 6 wherein the compliant conduits are detachably coupled to the treatment controller.

8. The modular low level light therapy system of claim 1 further comprising at least one power source in communication with the device circuit of the treatment controller.

9. The modular low level light therapy system of claim 1 wherein a first device circuit is in communication with and configured to provide data to and receive data from at least one a second device circuit positioned on the body of the patient.

10. The modular low level light therapy system of claim 1 at least one device circuits positioned on the body of the patient are in communication with at least one additional device circuit positioned on the body of the patient, wherein the multiple device circuits form a distributed control system architecture.

11. The modular low level light therapy system of claim 1 wherein at least one device circuit positioned on the body of the patient is in communication with at least one external controller.

12. The modular low level light therapy system of claim 11 wherein the external controller comprises at least one device selected from the group consisting of
smartphones, handheld devices, computers, computer networks, and processors.

13. The modular low level light therapy system of claim 1 wherein device circuit includes at least one device or system selected from the group consisting of semiconductor devices, chips, sensors, controllers, processors, power supplies, batteries, energy sources, voltage regulators, current regulators, user interfaces, display devices, communication devices, user interfaces, wireless devices, MEMs devices, lab-on-a-chip systems, GPS devices, tactile alert generators, audio alert devices, and signaling devices.

14. The modular low level light therapy system of claim 1 wherein at least one of the sensors is selected from the group consisting of thermocouples, accelerometers, heart rate sensors, blood pressure sensors, respiration sensors, EEG devices, ECG devices, flow sensors, oxygenation sensors, cryotherapy systems, tissue temperature sensors, biomedical sensors, chemical sensors, small molecule sensors, ion sensitive devices and materials, protein sensing devices and materials, lipid sensing devices and materials, and force meters.

15. A modular low level light therapy system having a distributed control architecture, comprising:
two or more device bodies configured to be positioned on the body of a user proximate to a treatment area;
one or more light sources positioned on each device body, the light sources configured to output one or more therapeutic optical signals to the treatment area, the light sources comprising of an array of multiple semiconductor emitters;
one or more sensors configured to measure one or more treatment parameter from an area of interest of a body of a patient; and
one or more treatment controllers positioned on each device body, the treatment controllers having at least one device circuit therein, the device circuits in communication with the light sources and device circuits positioned proximate thereto, the device circuits configured to provide at least one control signal to the light sources to regulate the operation of at least one emitter of the light sources to vary the operation of the light sources based on data received from the sensors thereby forming a distributed control architecture.

16. The modular low level light therapy system of claim 15 wherein at least one device body comprises a compliant substrate configured to engage and retain at least of the light source and treatment controller therein.

17. The modular low level light therapy system of claim 15 wherein at least one light source comprises one or more devices selected from the group consisting of LEDs, super luminescent LEDs, laser diodes, edge-emitting laser devices, vertical cavity surface emitting laser devices, and fiber optic devices.

18. The modular low level light therapy system of claim 15 further comprising multiple light sources positioned on one or more compliant conduits, each conduit coupled to a body of the treatment controller.

19. The modular low level light therapy system of claim 15 wherein the distributed control architecture is in communication with at least one external device selected from the group consisting of smartphones, handheld devices, computers, computer networks, and processors.

20. The modular low level light therapy system of claim 15 wherein at least one device circuit includes at least one device or system selected from the group consisting of semiconductor devices, chips, sensors, controllers, processors, power supplies, batteries, energy sources, voltage regulators, current regulators, user interfaces, display devices, communication devices, user interfaces, wireless devices, MEMs devices, lab-on-a-chip systems, GPS devices, tactile alert generators, audio alert devices, and signaling devices.

21. The modular low level light therapy system of claim 15 wherein at least one of the sensors is selected from the group consisting of thermocouples, accelerometers, heart rate sensors, blood pressure sensors, respiration sensors, EEG devices, ECG devices, flow sensors, oxygenation sensors, cryotherapy systems, tissue temperature sensors, biomedical sensors, chemical sensors, small molecule sensors, ion sensitive devices and materials, protein sensing devices and materials, lipid sensing devices and materials, and force meters.
